(19) United States
(12) Patent Application Publication
(10) Pub. No.: US 2025/0264379 A1
(43) Pub. Date: Aug. 21, 2025

(54) BIOMETRIC SENSING DEVICE

(71) Applicant: ALTEK BIOTECHNOLOGY CORPORATION, Hsinchu City (TW)

(72) Inventors: Jung-Fu Chien, Hsinchu City (TW); Chun-Ming Hu, Hsinchu City (TW)

(21) Appl. No.: 18/582,607

(22) Filed: Feb. 20, 2024

(51) Int. Cl. G01N 1/10; G01N 33/66; G01R 1/04

(52) U.S. Cl. CPC G01N 1/10; G01R 1/04; G01N 33/66

(57) ABSTRACT

A biometric sensing device of preventing circuit failure includes a case, a circuit board, a cover, an operation component and an auxiliary component. The case includes a retaining wall and a piercing hole structure. The circuit board is disposed inside the case and includes an electronic switch. The cover is disposed above the case and includes an opening structure. The operation component is movably disposed on the case and protruded through the opening structure. The auxiliary component is connected to the operation component and disposed under the case. The auxiliary component includes a main body and a blocking portion. The main body is divided into a central region and an outer region adjacent to each other, and the operation component passes through the central region. The blocking portion disposed on the outer region and around the operation component, and adapted to clog liquid flowing into the piercing hole structure.

### BACKGROUND OF THE INVENTION

### 1. Field of the Invention

The present invention relates to a biometric sensing device, and more particularly, to a biometric sensing device for circuit failure prevention.

### 2. Description of the Prior Art

In the medical institutions, a blood glucose meter may be the shared medical equipment among several patients. For avoiding bacterial cross-infection caused by the shared the medical equipment, the blood glucose meter must comply with the disinfection principles required by health regulations. The conventional blood glucose meter must be disinfected by disinfection wipes specified by the medical equipment manufacturer; however, when the blood glucose meter is cleaned for a certain number of times, the conventional blood glucose meter may have drawbacks of the blood glucose detection error or the function failure, which is resulted from the disinfectant leaking into the blood glucose meter. Therefore, design of a blood glucose meter of preventing the disinfectant from leaking into the internal circuit board is an important issue in the medical equipment industry.

### SUMMARY OF THE INVENTION

The present invention provides a biometric sensing device for circuit failure prevention for solving above drawbacks.

According to the claimed invention, a biometric sensing device of preventing circuit failure includes a case, a circuit board, a cover and an operation component. The case includes a retaining wall and a piercing hole structure, and the piercing hole structure is surrounded by the retaining wall. The circuit board is disposed inside the case and includes an electronic switch. The cover is disposed above the case and comprising an opening structure. The operation component is movably disposed on the case, and an accommodating chamber is formed between the case, the cover and the operation component. The operation component includes a pressing portion, an actuating portion, a supporting portion and an abutting portion. The pressing portion is protruded through the opening structure. The actuating portion is connected to a bottom of the pressing portion, and adapted to inset into the piercing hole structure for detachably contacting the electronic switch. The supporting portion is connected to a lateral side of the pressing portion, and located between the cover and the case to support the pressing portion. The abutting portion is connected to the pressing portion and located between the actuating portion and the supporting portion. The abutting portion slidably abuts against the retaining wall to prevent liquid in the accommodating chamber from flowing into the piercing hole structure through a first gap formed between the abutting portion and the retaining wall.

According to the claimed invention, a second gap is formed between the operation component and the cover, and a structural width of the second gap is greater than a structural width of the first gap. The abutting portion includes a first surface and a second surface opposite to each other, the first surface slidably abuts against the retaining wall, the second surface is adapted to push the liquid to drain out of the accommodating chamber through the second gap in accordance with a movement of the pressing portion.

According to the claimed invention, the case further includes a plate body where on the retaining wall is disposed, and a distance between a top end of the retaining wall and the plate body is greater than a distance between the cover and the plate body. The supporting portion is made of resilient and deformable material, and comprises a first end and a second end opposite to each other, the first end abuts against the plate body, the second case contacts or is separated from the cover in accordance with a movement of the pressing portion. The operation component further includes a positioning portion disposed on the first end and fixed to the case for constraining rotation of the pressing portion.

According to the claimed invention, the biometric sensing device further includes an auxiliary component connected to the operation component and disposed under the case, for clogging and diverting the liquid. The auxiliary component includes a main body divided into a central region and an outer region adjacent to each other, the actuating portion is connected to the central region. The auxiliary component further includes a blocking portion disposed on the outer region and around the actuating portion.

According to the claimed invention, a top end of the blocking portion abuts against a lower surface of the case. The blocking portion is a closed ring structure or a non-closed C-shaped structure. The case further includes an engaging portion disposed on a lower surface of the case and adapted to engage with a top end of the blocking portion. The auxiliary component further includes a guiding portion disposed on the outer region and around the blocking portion. The guiding portion is extended toward a position located above a drain hole formed on the case.

According to the claimed invention, a biometric sensing device of preventing circuit failure includes a case, a circuit board, a cover, an operation component and an auxiliary component. The case includes a retaining wall and a piercing hole structure, and the piercing hole structure is surrounded by the retaining wall. The circuit board is disposed inside the case and includes an electronic switch. The cover is disposed above the case and includes an opening structure. The operation component is movably disposed on the case and protruded through the opening structure, and the operation component partly passes into the piercing hole structure for detachably contacting the electronic switch. The auxiliary component is connected to the operation component and disposed under the case. The auxiliary component includes a main body and a blocking portion. The main body is divided into a central region and an outer region adjacent to each other, and the operation component passes through the central region. The blocking portion disposed on the outer region and around the operation component, and adapted to clog liquid flowing into the piercing hole structure.

According to the claimed invention, the retaining wall includes an inner surface adjacent to the operation component, and a first included angle between a tangent line of the inner surface and a planar normal vector of the plate body is smaller than five degrees. The retaining wall further includes an outer surface opposite to the operation component, and a second included angle between a tangent line of the outer surface and the planar normal vector is greater than the first included angle.

The biometric sensing device of the present invention can be applied to a blood glucose meter or any similar product, and can optionally utilize the operation component or the auxiliary component with the specific design, or simultaneously utilize the operation component and the auxiliary component with the specific design, to prevent the liquid outside the case from flowing into the gap between the cover and the operation component; if the liquid flows into the gap between the cover and the operation component, the liquid can be accumulated inside the accommodating chamber formed between the case, the cover and the operation component, and the second surface of the abutting portion can be used to drain the accumulated liquid out of the accommodating chamber when pressing the operation component; if the accumulated liquid flows from the accommodating chamber into the piercing hole structure, the blocking portion of the auxiliary component can avoid the liquid from flowing towards the circuit board, and the guiding portion of the auxiliary component can guide the liquid flowing across the blocking portion to flow towards the drain hole for being drained out of the case. The biometric sensing device of the present invention can provide liquid blocking and diversion ways and prevent the liquid outside the case from flowing onto the circuit board inside the case to result in the circuit failure.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 and FIG. 2 are diagrams of a biometric sensing device in different views according to an embodiment of the present invention.

FIG. 3 is an exploded diagram of the biometric sensing device according to a first embodiment of the present invention.

FIG. 4 is a sectional view of some part of the biometric sensing device according to the first embodiment of the present invention.

FIG. 5 is a diagram of an operation component according to the first embodiment of the present invention.

FIG. 6 is a diagram of an auxiliary component according to the first embodiment of the present invention.

FIG. 7 is a sectional view of the biometric sensing device in another operation mode according to the first embodiment of the present invention.

FIG. 8 is a sectional view of some part of the biometric sensing device according to a second embodiment of the present invention.

FIG. 9 is a sectional view of some part of the biometric sensing device according to a third embodiment of the present invention.

FIG. 10 is an enlarged diagram of the biometric sensing device according to the third embodiment of the present invention.

### DETAILED DESCRIPTION

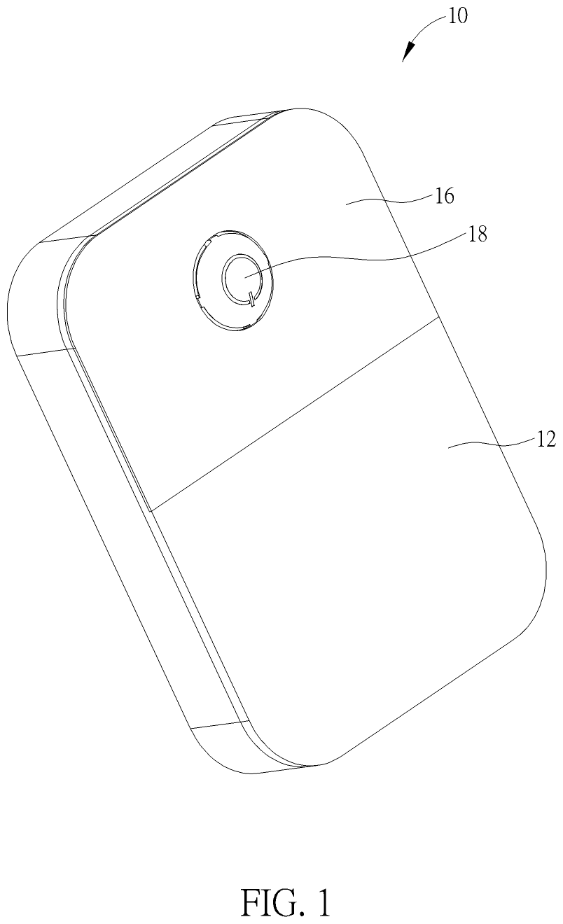

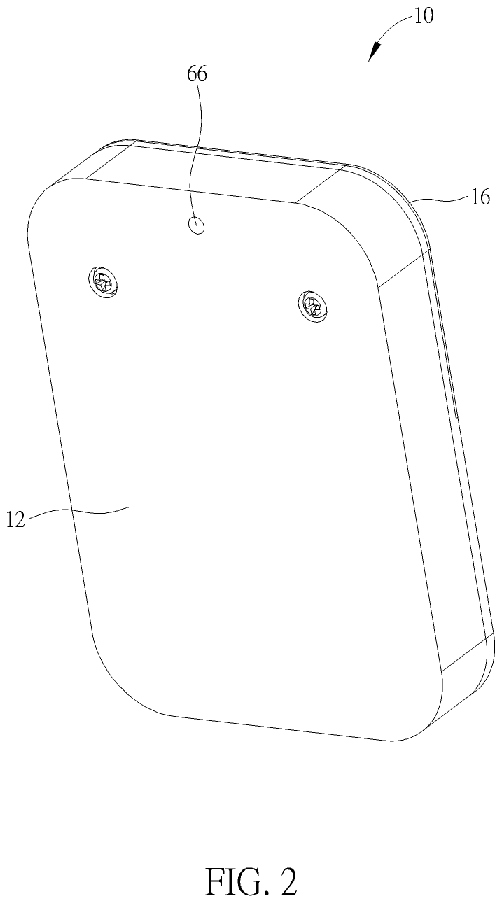

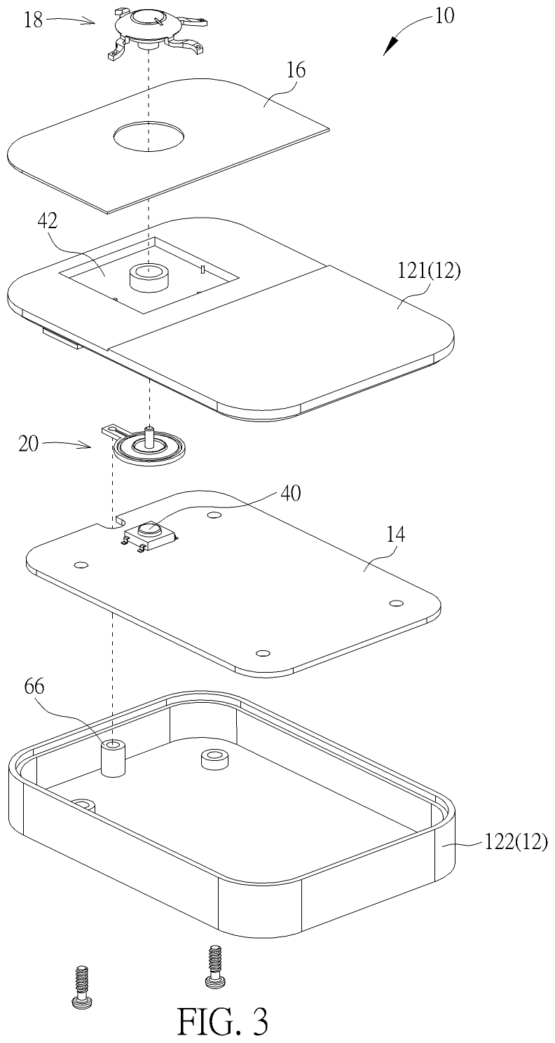

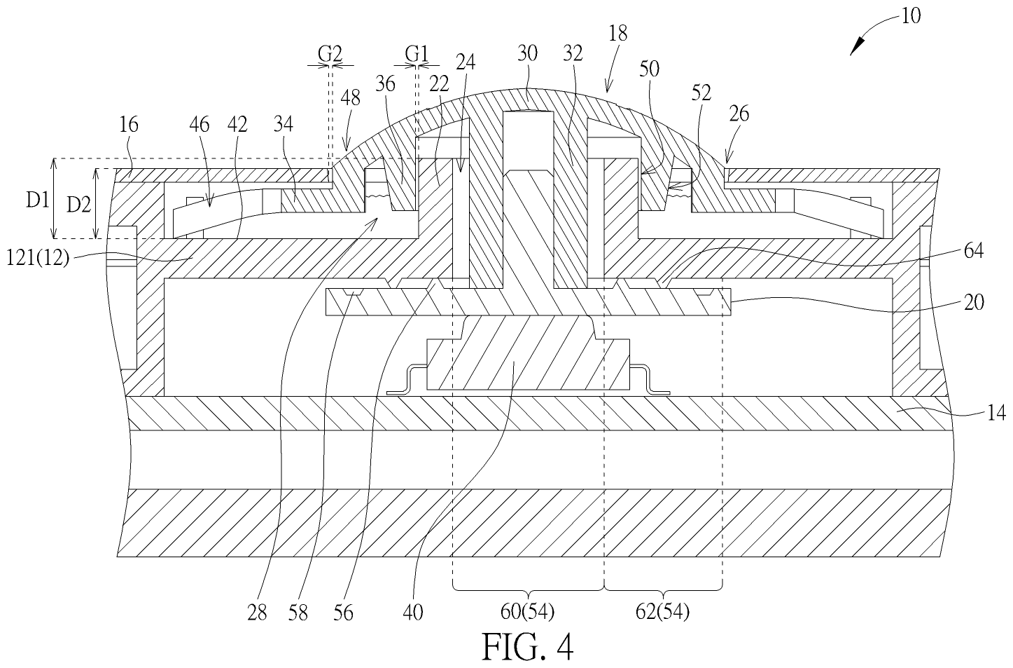

Please refer to FIG. 1 to FIG. 4. FIG. 1 and FIG. 2 are diagrams of a biometric sensing device 10 in different views according to an embodiment of the present invention. FIG. 3 is an exploded diagram of the biometric sensing device 10 according to a first embodiment of the present invention. FIG. 4 is a sectional view of some part of the biometric sensing device 10 according to the first embodiment of the present invention. The biometric sensing device 10 can include a case 12, a circuit board 14, a cover 16, an operation component 18 and an auxiliary component 20. The case 12 can have an upper housing 121 and a lower housing 122. The circuit board 14 can be disposed inside the case 12, such as being located on the lower housing 122. The cover 16 can be disposed above the upper housing 121. The operation component 18 can be movably disposed on the upper housing 121, and be partly protruded from the cover 16. The auxiliary component 20 can be connected to the operation component 18, and be disposed under the upper housing 121.

The operation component 18 can be used to drain liquid accumulated between the upper housing 121, the cover 16 and the operation component 18 out of the case 12. The auxiliary component 20 can prevent the liquid from flowing onto the circuit board 14, and further can drain the liquid flowing into the case 12 out of the biometric sensing device 10. The upper housing 121 of the case 12 can include a retaining wall 22 and a piercing hole structure 24. The piercing hole structure 24 can be surrounded by the retaining wall 22. The cover 16 can include an opening structure 26; the cover 16 can be disposed above the upper housing 121 by putting the opening structure 26 around the retaining wall 22 and the piercing hole structure 24. The operation component 18 can be partly inserted into a gap between the case 12 and the cover 16, and a part of the operation component 18 can cover the retaining wall 22 and the piercing hole structure 24 to protrude from the opening structure 26. An accommodating chamber 28 can be formed between the case 12, the cover 16 and the operation component 18. The auxiliary component 20 can be disposed under the retaining wall 22 and the piercing hole structure 24.

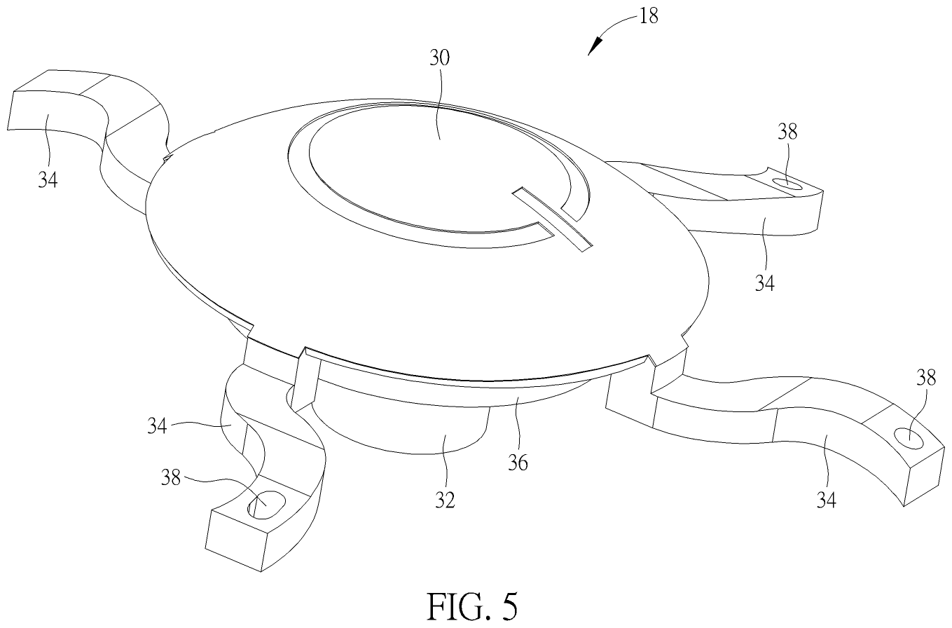

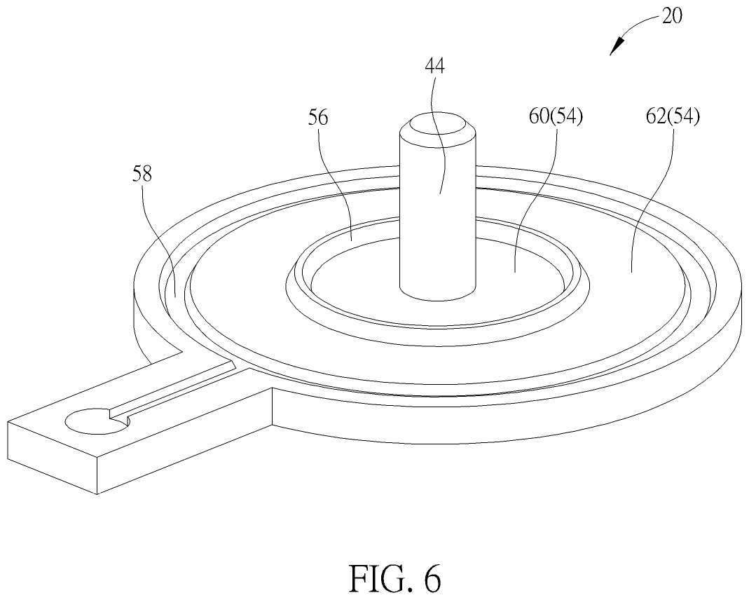

Please refer to FIG. 4 to FIG. 6. FIG. 5 is a diagram of the operation component 18 according to the first embodiment of the present invention. FIG. 6 is a diagram of the auxiliary component 20 according to the first embodiment of the present invention. The operation component 18 can include a pressing portion 30, an actuating portion 32, a supporting portion 34, an abutting portion 36 and a positioning portion 38. The pressing portion 30 can be protruded through the opening structure 26 of the cover 16, and a symbol can be printed on the pressing portion 30 to be touched and identified by a user. The actuating portion 32 can be connected to a bottom of the pressing portion 30, and can be inserted into the piercing hole structure 24 of the case 12, and may contact against an electronic switch 40 of the circuit board 14 via the auxiliary component 20 in a detachable manner or in a connective manner. The supporting portion 34 can be connected to a lateral side of the pressing portion 30, and can be extended into the gap between the cover 16 and the case 12 for supporting the pressing portion 30. The abutting portion 36 can be connected to the pressing portion 30 and located between the actuating portion 32 and the supporting portion 34, and can further abut against the retaining wall 22 of the case 12 in a slidable manner.

The upper housing 121 of the case 12 can further include a plate body 42. An outer surface of the plate body 42 opposite to the upper housing 121 can be formed as a concave structure, as the embodiment shown in FIG. 3; the gap between the plate body 42 and the cover 16 can accommodate the supporting portion 34 of the operation component 18. In addition, the piercing hole structure 24 can be formed on a central area of the plate body 42, and the retaining wall 22 can be disposed on the plate body 42 to surround the piercing hole structure 24. A structural height of the retaining wall 22 can be preferably greater/higher than the opening structure 26 of the cover 16, which means a distance D1 between a top end of the retaining wall 22 and the plate body 42 can be greater than a distance D2 between the cover 16 and the plate body 42.

The actuating portion 32 can be disposed on a connecting portion 44 of the auxiliary component 20, so as to ensure that the auxiliary component 20 can be synchronously moved in the same direction in accordance with pressing motion of the operation component 18. The supporting portion 34 preferably can be made of resilient and deformable material, and include a first end 46 and a second end 48 opposite to each other. The first end 46 can abut against the plate body 42; the second end 48 can be connected to the pressing portion 30, and may abut against or be separated from the cover 16 in accordance with motion of the pressing portion 30. The positioning portion 38 of the operation component 18 can be disposed on the first end 46 of the supporting portion 34, and be used to fix onto the upper housing 121 of the case 12 for constraining rotation of the pressing portion 30. For example, the positioning portion 38 can be fixed onto the upper housing 121 in an adhering manner, in an engaging manner or in a locking manner, and variation of the positioning portion 38 can depend on a design demand.

The abutting portion 36 can include a first surface 50 and a second surface 52 opposite to each other. The first surface 50 can slidably abut against the retaining wall 22; generally, the first surface 50 can be a flat surface close to the retaining wall 22. The second surface 52 can be an inclined surface relative to the first surface 50, which means a second planar normal vector of the second surface 52 can be interleaved with a first planar normal vector of the first surface 50, and an included angle between the first planar normal vector and the second planar normal vector may be approximately forty-five degree, with an allowable error of plus or minus fifteen percent. Besides, a first gap G1 can be formed between the first surface 50 of the abutting portion 36 and the retaining wall 22, and a second gap G2 can be formed between the operation component 18 (such as a junction of the pressing portion 30 and the supporting portion 34) and the cover 16; a structural width of the second gap G2 can be greater than a structural width of the first gap G1.

The auxiliary component 20 can include a connecting portion 44, a main body 54, a blocking portion 56 and a guiding portion 58. The main body 54 can be divided into a central region 60 and an outer region 62 adjacent to each other. The connecting portion 44 can be disposed on the central region 60, and the actuating portion 32 of the operation component 18 can be disposed on the connecting portion 44 to connect with the central region 60 in the detachable manner or in the connective manner. The blocking portion 56 can be disposed on the outer region 62 to surround the connecting portion 44, and further surround the actuating portion 32 disposed on the connecting portion 44. The blocking portion 56 can be a protruding structure formed on the main body 54; a top end of the blocking portion 56 can preferably abut against a lower surface of the upper housing 121 of the case 12, or be indicated as abutting against a lower surface of the plate body 42. The upper housing 121 of the case 12 can include an engaging portion 64 disposed on the lower surface of the upper housing 121; the engaging portion 64 can be engaged with the top end of the blocking portion 56, or abut against the main body 54 of the auxiliary component 20 and be located adjacent to the blocking portion 56. The guiding portion 58 can be disposed on the outer region 62 and surround the blocking portion 56; the guiding portion 58 can be extended to a position above a drain hole 66 formed on the case 12.

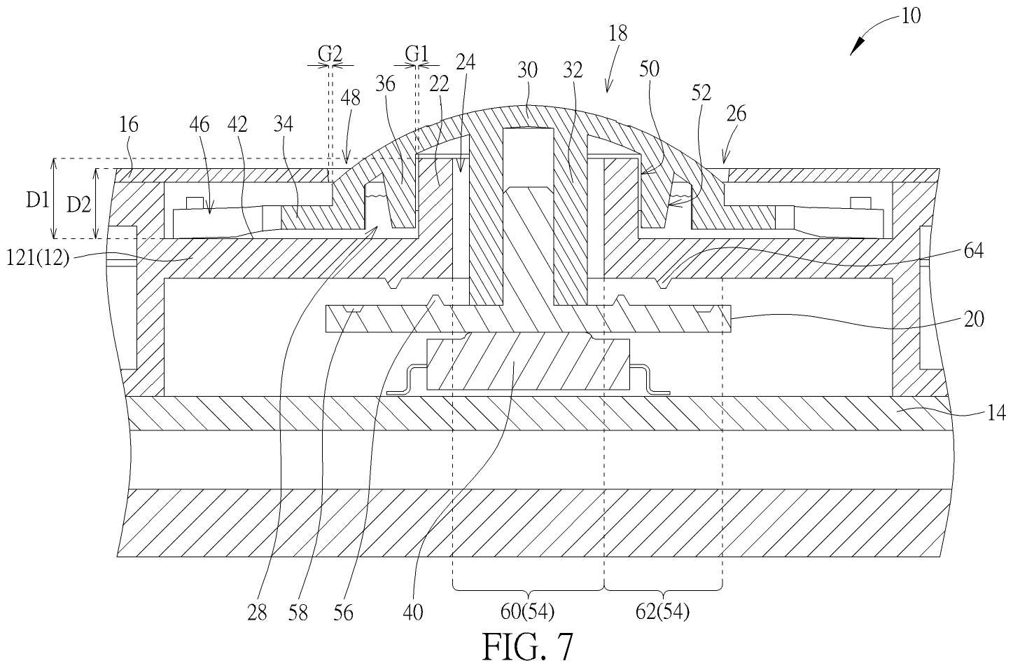

Please refer to FIG. 4 to FIG. 7. FIG. 7 is a sectional view of the biometric sensing device 10 in another operation mode according to the first embodiment of the present invention. As shown in FIG. 4, the operation component 18 is not pressed and located on an initial position; the first gap G1 may be formed between the operation component 18 and the case 12 due to an assembly demand, and the second gap G2 may be formed between the operation component 18 and the cover 16 due to the assembly demand. The liquid outside the biometric sensing device 10 may flow into the second gap G2 and be accumulated inside the accommodating chamber 28. As shown in FIG. 7, when the operation component 18 is pressed and moved downwardly, the actuating portion 32 can be accordingly moved to actuate the electronic switch 40 of the circuit board 14 via the auxiliary component 20; in the meantime, the supporting portion 34 can be forced and resiliently deformed, and the first surface 50 of the abutting portion 36 can slidably abut against the retaining wall 22 to prevent the liquid from flowing into the piercing hole structure 24 through the first gap G1, and the second surface 50 of the abutting portion 36 can push the liquid accumulated inside the accommodating chamber 28 towards the second gap G2 for draining out of the accommodating chamber 28. When an external force applied to the operation component 18 is removed, a resilient recovering force of the supporting portion 34 can move the operation component 18 back to the initial position.

Moreover, the biometric sensing device 10 can further utilize the auxiliary component 20 to block and divert the liquid flowing into the piercing hole structure 24. As mentioned above, the biometric sensing device 10 can utilize specific design of the operation component 18 to drain the liquid out of the accommodating chamber 28, but the liquid may still flow into the piercing hole structure 24; for preventing the liquid inside the piercing hole structure 24 from flowing towards the circuit board 14 to result in the circuit failure, the biometric sensing device 10 can dispose the auxiliary component 20 under the piercing hole structure 24 of the case 12. The blocking portion 56 of the auxiliary component 20 can be a closed ring structure, as shown in FIG. 6; or the blocking portion 56 can be a non-closed C-shaped structure, which is a modified form of the closed ring structure being partly cut. The blocking portion 56 of the auxiliary component 20 and/or the engaging portion 64 of the case 12 can avoid the liquid inside the piercing hole structure 24 from flowing towards the circuit board 14. If the liquid still flows across the blocking portion 56, the auxiliary component 20 can further utilize the guiding portion 58 to guide the liquid to flow towards the drain hole 66 of the case 12, so as to drain the liquid out of the case 12 without the circuit failure.

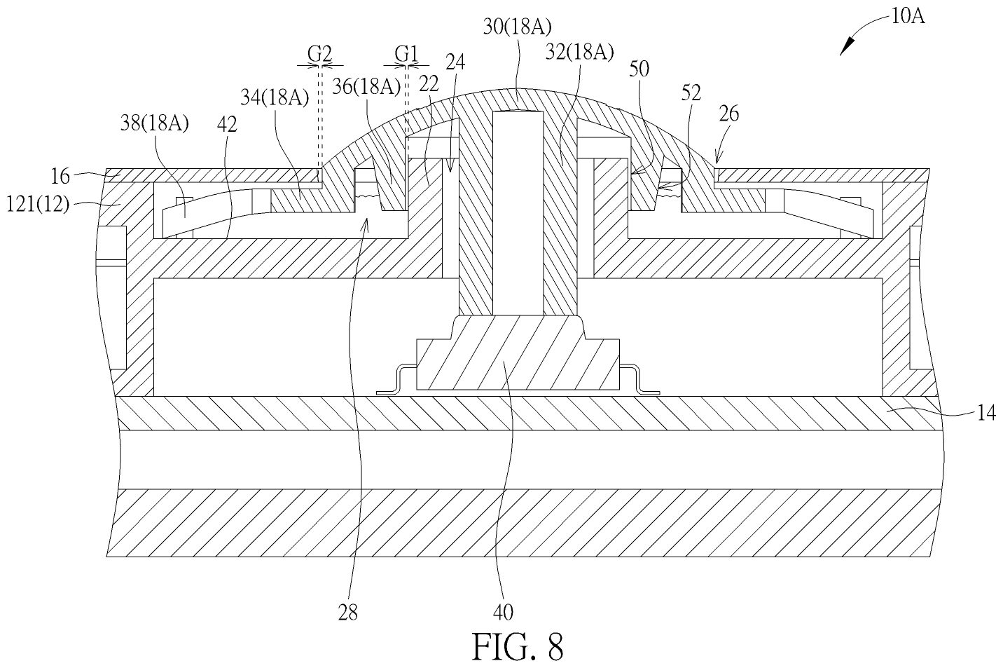

Please refer to FIG. 1, FIG. 2 and FIG. 8. FIG. 8 is a sectional view of some part of the biometric sensing device 10A according to a second embodiment of the present invention. The biometric sensing device 10A can include the case 12, the circuit board 14, the cover 16, and the operation component 18A. The case 12 can have the upper housing 121 and the lower housing 122 assembled with each other. The circuit board 14 can be disposed inside the case 12, such as being located on the lower housing 122. The cover 16 can be disposed above the upper housing 121. The operation component 18A can be movably disposed on the upper housing 121, and be partly protruded from the cover 16. The upper housing 121 of the case 12 can include the retaining wall 22 and the piercing hole structure 24. The piercing hole structure 24 can be surrounded by the retaining wall 22. The cover 16 can include the opening structure 26; the cover 16 can be disposed above the upper housing 121 by putting the opening structure 26 around the retaining wall 22 and the piercing hole structure 24. The operation component 18A can be partly inserted into the gap between the case 12 and the cover 16, and some part of the operation component 18A can cover the retaining wall 22 and the piercing hole structure 24 to protrude from the opening structure 26. The accommodating chamber 28 can be formed between the case 12, the cover 16 and the operation component 18A.

The operation component 18A can include the pressing portion 30, the actuating portion 32, the supporting portion 34, the abutting portion 36 and the positioning portion 38. The pressing portion 30 can be protruded through the opening structure 26 of the cover 16, and the symbol can be printed on the pressing portion 30 to be touched and identified by the user. The actuating portion 32 can be connected to the bottom of the pressing portion 30, and can be inserted into the piercing hole structure 24 of the case 12, and may actuate the electronic switch 40 of the circuit board 14 in the detachable manner or in the connective manner. The supporting portion 34 can be connected to the lateral side of the pressing portion 30, and can be extended into the gap between the cover 16 and the case 12 for supporting the pressing portion 30. The abutting portion 36 can be connected to the pressing portion 30 and located between the actuating portion 32 and the supporting portion 34, and can further abut against the retaining wall 22 of the case 12 in the slidable manner. The upper housing 121 of the case 12 can further include the plate body 42; the gap between the plate body 42 and the cover 16 can accommodate the supporting portion 34 of the operation component 18A. In addition, the piercing hole structure 24 can be formed on the central area of the plate body 42, and the retaining wall 22 can be disposed on the plate body 42 to surround the piercing hole structure 24. The structural height of the retaining wall 22 can be preferably greater or higher than the opening structure 26 of the cover 16.

The actuating portion 32 can be extended into the upper housing 121 for directly contacting the electronic switch 40; the actuating portion 32 can be synchronously moved in the same direction in accordance with the pressing motion of the operation component 18A for switching on or off the electronic switch 40. The supporting portion 34 preferably can be made of resilient and deformable material, and may abut against or be separated from the cover 16 in accordance with the motion of the pressing portion 30. The positioning portion 38 of the operation component 18A can be disposed on the free end of the supporting portion 34, and be used to fix onto the upper housing 121 of the case 12 for constraining the rotation of the pressing portion 30. The abutting portion 36 can include the first surface 50 and the second surface 52 opposite to each other. The first surface 50 can be the flat surface close to the retaining wall 22 for slidably abutting against the retaining wall 22, and the second surface 52 can be the inclined surface relative to the first surface 50. Besides, the first gap G1 can be formed between the first surface 50 of the abutting portion 36 and the retaining wall 22, and the second gap G2 can be formed between the operation component 18A (such as the junction of the pressing portion 30 and the supporting portion 34) and the cover 16; the structural width of the second gap G2 can be greater than the structural width of the first gap G1.

Due to the second gap G2 between the operation component 18A and the cover 16, the liquid outside the biometric sensing device 10A may flow into the second gap G2 and be accumulated inside the accommodating chamber 28. In the meantime, the user can press the operation component 18A for downward motion, and the actuating portion 32 can be moved downwardly and accordingly to actuate the electronic switch 40 of the circuit board 14; the first surface 50 of the abutting portion 36 can slidably abut against the retaining wall 22 to prevent the liquid from flowing into the piercing hole structure 24 through the first gap G1, and the second surface 50 of the abutting portion 36 can push the liquid accumulated inside the accommodating chamber 28 towards the second gap G2 for draining out of the accommodating chamber 28. In other possible embodiments, a waterproof gasket or any similar element (which is not shown in the figures) can be optionally disposed between the retaining wall 22 and the abutting portion 36, and can be used to avoid the liquid accumulated inside the accommodating chamber 28 from flowing into the piercing hole structure 24 through the first gap G1.

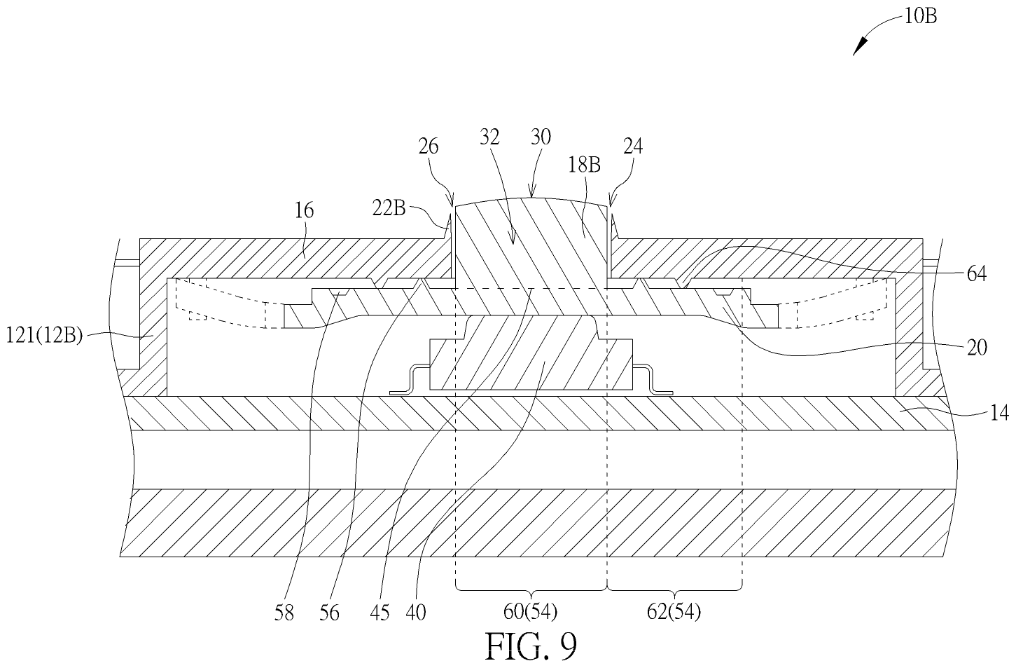

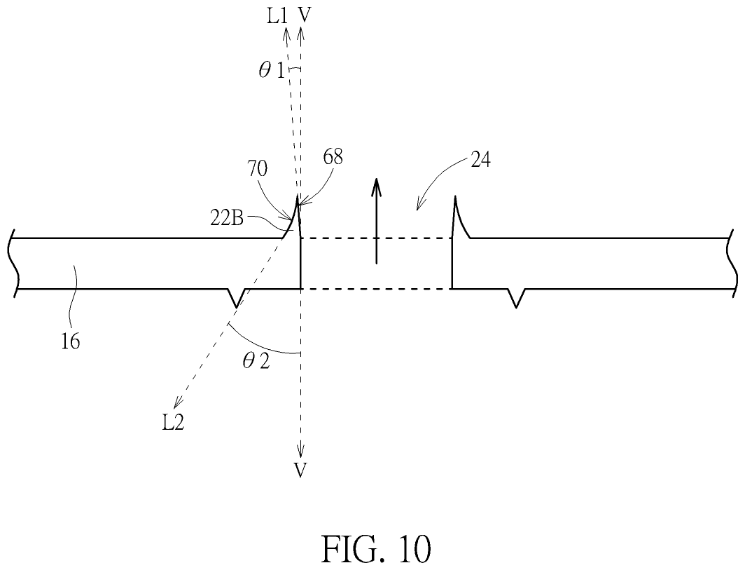

Please refer to FIG. 1, FIG. 2, FIG. 9 and FIG. 10. FIG. 9 is a sectional view of some part of the biometric sensing device 10B according to a third embodiment of the present invention. FIG. 10 is an enlarged diagram of the biometric sensing device 10B according to the third embodiment of the present invention. The biometric sensing device 10B can include the case 12B, the circuit board 14, the cover 16, the operation component 18B and the auxiliary component 20. The case 12B can have the upper housing 121 and the lower housing 122 assembled with each other. The circuit board 14 can be disposed inside the case 12B, such as being located on the lower housing 122. The cover 16 can be disposed above the upper housing 121. The operation component 18B can be movably disposed on the upper housing 121, and be partly protruded from the cover 16. The auxiliary component 20 can be connected to the operation component 18B, and be disposed inside the upper housing 121. The upper housing 121 of the case 12B can include the retaining wall 22B and the piercing hole structure 24. The piercing hole structure 24 can be surrounded by the retaining wall 22B. The cover 16 can include the opening structure 26; the cover 16 can be disposed one the upper housing 121 by corresponding the opening structure 26 to the piercing hole structure 24. The operation component 18B can be partly inserted into the piercing hole structure 24 of the case 12B and/or the opening structure 26 of the cover 16 to connect with the auxiliary component 20; for example, the operation component 18B can be monolithically integrated with the auxiliary component 20, which depends on the design demand. A part of the operation component 18B can protrude from the piercing hole structure 24 of the case 12B and/or the opening structure 26 of the cover 16 for being pressed by the user. The auxiliary component 20 can be disposed under the retaining wall 22B and the piercing hole structure 24, and may be monolithically integrated with the operation component 18B, or may further be detachably connected with the operation component 18B.

The pressing portion 30 (such as a top surface) of the operation component 18B can protrude from the piercing hole structure 24 of the case 12B and/or the opening structure 26 of the cover 16, and the symbol can be printed on the pressing portion 30 to be touched and identified by the user. The actuating portion 32 (such as a middle section) of the operation component 18B can be extended from the pressing portion 30 downwardly, and may contact against the electronic switch 40 of the circuit board 14 via the auxiliary component 20 in the detachable manner or in the connective manner. The supporting portion (which is not marked in FIG. 9) of the operation component 18B can be connected to the lateral side of the pressing portion 30 or the lateral side of the auxiliary component 20 (such as an element drawn as a dotted line in FIG. 9), and can be disposed on the cover 16 and/or the case 12B for supporting the pressing portion 30. In addition, the piercing hole structure 24 can be formed on the central area of the upper housing 121, and the retaining wall 22B can be disposed on a position of the upper housing 121 surrounding the piercing hole structure 24. The actuating portion 32 can be disposed on the central position area 45 of the auxiliary component 20, so as to ensure that the auxiliary component 20 can be synchronously moved in the same direction in accordance with the pressing motion of the operation component 18B. The supporting portion (which is not marked in FIG. 9) of the operation component 18B preferably can be made of resilient and deformable material, and may abut against or be separated from the cover 16 and/or the casing 12B in accordance with motion of the pressing portion 30. The positioning portion (which is not marked in FIG. 9) of the operation component 18B can be disposed on the free end of the supporting portion (which is not marked in FIG. 9), and be used to fix onto the case 12B and/or the cover 16 for constraining the rotation of the pressing portion 30.

The auxiliary component 20 can include the central position area 45, the main body 54, the blocking portion 56 and the guiding portion 58. The main body 54 can be divided into the central region 60 and the outer region 62 adjacent to each other. The central position area 45 can correspond to the central region 60, and the actuating portion 32 of the operation component 18B can be connected with the central region 60. As mentioned above, the operation component 18B and the auxiliary component 20 may be monolithically integrated with each other in a preferable manner. The blocking portion 56 can be disposed on the outer region 62 to surround the central region 60, and further surround the actuating portion 32 disposed on the central region 60. The blocking portion 56 can be the protruding structure formed on the main body 54; the top end of the blocking portion 56 can preferably abut against the lower surface of the upper housing 121 of the case 12B. The upper housing 121 of the case 12B or the cover 16 can include the engaging portion 64 disposed on the lower surface of the upper housing 121 or the cover 16; the engaging portion 64 can be engaged with the top end of the blocking portion 56, or abut against the main body 54 of the auxiliary component 20 and be located adjacent to the blocking portion 56. The guiding portion 58 can be disposed on the outer region 62 and surround the blocking portion 56; the guiding portion 58 can be extended to the position above the drain hole 66 formed on the case 12.

As shown in FIG. 10, it should be mentioned that the retaining wall 22B can include an inner surface 68 adjacent to the operation component 18B, and a first included angle θ1 between a tangent line L1 of the inner surface 68 and a planar normal vector V of the cover 16 can be smaller than five degrees; besides, the retaining wall 22B can further include an outer surface 70 opposite to the operation component 18B, and a second included angle θ2 between a tangent line L2 of the outer surface 70 and the planar normal vector V can be greater than the first included angle θ1. Therefore, the retaining wall 22B can block the liquid flowing through the gap between the operation component 18B and the case 12B, to reduce the amount of the liquid flowing towards the auxiliary component 20, and the blocking portion 56 and the guiding portion 58 of the auxiliary component 20 can rapidly and effectively drain the liquid out of the drain hole 66 of the case 12B.

In conclusion, the biometric sensing device of the present invention can be applied to a blood glucose meter or any similar product, and can optionally utilize the operation component or the auxiliary component with the specific design, or simultaneously utilize the operation component and the auxiliary component with the specific design, to prevent the liquid outside the case from flowing into the gap between the cover and the operation component; if the liquid flows into the gap between the cover and the operation component, the liquid can be accumulated inside the accommodating chamber formed between the case, the cover and the operation component, and the second surface of the abutting portion can be used to drain the accumulated liquid out of the accommodating chamber when pressing the operation component; if the accumulated liquid flows from the accommodating chamber into the piercing hole structure, the blocking portion of the auxiliary component can avoid the liquid from flowing towards the circuit board, and the guiding portion of the auxiliary component can guide the liquid flowing across the blocking portion to flow towards the drain hole for being drained out of the case. Comparing to the prior art, the biometric sensing device of the present invention can provide liquid blocking and diversion ways and prevent the liquid outside the case from flowing onto the circuit board inside the case to result in the circuit failure.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A biometric sensing device of preventing circuit failure, comprising:
a case comprising a retaining wall and a piercing hole structure, the piercing hole structure being surrounded by the retaining wall;
a circuit board disposed inside the case and comprising an electronic switch;
a cover disposed above the case and comprising an opening structure; and
an operation component movably disposed on the case, an accommodating chamber being formed between the case, the cover and the operation component, the operation component comprising:
a pressing portion protruded through the opening structure;
an actuating portion connected to a bottom of the pressing portion, and adapted to inset into the piercing hole structure for detachably contacting the electronic switch;
a supporting portion connected to a lateral side of the pressing portion, and located between the cover and the case to support the pressing portion; and
an abutting portion connected to the pressing portion and located between the actuating portion and the supporting portion, the abutting portion slidably abutting against the retaining wall to prevent liquid in the accommodating chamber from flowing into the piercing hole structure through a first gap formed between the abutting portion and the retaining wall.

2. The biometric sensing device of claim 1, wherein a second gap is formed between the operation component and the cover, and a structural width of the second gap is greater than a structural width of the first gap.

3. The biometric sensing device of claim 2, wherein the abutting portion comprising a first surface and a second surface opposite to each other, the first surface slidably abuts against the retaining wall, the second surface is adapted to push the liquid to drain out of the accommodating chamber through the second gap in accordance with a movement of the pressing portion.

4. The biometric sensing device of claim 1, wherein the case further comprises a plate body where on the retaining wall is disposed, and a distance between a top end of the retaining wall and the plate body is greater than a distance between the cover and the plate body.

5. The biometric sensing device of claim 4, wherein the supporting portion is made of resilient and deformable material, and comprises a first end and a second end opposite to each other, the first end abuts against the plate body, the second case contacts or is separated from the cover in accordance with a movement of the pressing portion.

6. The biometric sensing device of claim 5, wherein the operation component further comprises a positioning portion disposed on the first end and fixed to the case for constraining rotation of the pressing portion.

7. The biometric sensing device of claim 1, wherein the biometric sensing device further comprises an auxiliary component connected to the operation component and disposed under the case, for clogging and diverting the liquid.

8. The biometric sensing device of claim 7, wherein the auxiliary component comprises a main body divided into a central region and an outer region adjacent to each other, the actuating portion is connected to the central region.

9. The biometric sensing device of claim 8, wherein the auxiliary component further comprises a blocking portion disposed on the outer region and around the actuating portion.

10. The biometric sensing device of claim 9, wherein a top end of the blocking portion abuts against a lower surface of the case.

11. The biometric sensing device of claim 9, wherein the blocking portion is a closed ring structure or a non-closed C-shaped structure.

12. The biometric sensing device of claim 9, wherein the case further comprises an engaging portion disposed on a lower surface of the case and adapted to engage with a top end of the blocking portion.

13. The biometric sensing device of claim 8, wherein the auxiliary component further comprises a guiding portion disposed on the outer region and around the blocking portion.

14. The biometric sensing device of claim 13, wherein the guiding portion is extended toward a position located above a drain hole formed on the case.

15. A biometric sensing device of preventing circuit failure, comprising:
a case comprising a retaining wall and a piercing hole structure, the piercing hole structure being surrounded by the retaining wall;
a circuit board disposed inside the case and comprising an electronic switch;
a cover disposed above the case and comprising an opening structure;
an operation component movably disposed on the case and protruded through the opening structure, the operation component partly passing into the piercing hole structure for detachably contacting the electronic switch; and
an auxiliary component connected to the operation component and disposed under the case, the auxiliary component comprising:
a main body divided into a central region and an outer region adjacent to each other, the operation component passing through the central region; and
a blocking portion disposed on the outer region and around the operation component, and adapted to clog liquid flowing into the piercing hole structure.

16. The biometric sensing device of claim 15, wherein the auxiliary component further comprises a guiding portion disposed on the outer region and around the blocking portion, and adapted to divert the liquid passing through the blocking portion.

17. The biometric sensing device of claim 15, wherein a top end of the blocking portion abuts against a lower surface of the case.

18. The biometric sensing device of claim 15, wherein the blocking portion is a closed ring structure or a non-closed C-shaped structure.

19. The biometric sensing device of claim 15, wherein the case further comprises an engaging portion disposed on a lower surface of the case and adapted to engage with a top end of the blocking portion.

20. The biometric sensing device of claim 16, wherein the guiding portion is extended toward a position located above a drain hole formed on the case.

21. The biometric sensing device of claim 15, wherein the case further comprises a plate body, and the retaining wall is protruded from the plate body.

22. The biometric sensing device of claim 21, wherein the retaining wall comprises an inner surface adjacent to the operation component, and a first included angle between a tangent line of the inner surface and a planar normal vector of the plate body is smaller than five degrees.

23. The biometric sensing device of claim 22, wherein the retaining wall further comprises an outer surface opposite to the operation component, and a second included angle between a tangent line of the outer surface and the planar normal vector is greater than the first included angle.
